(19) United States
(12) Patent Application Publication
(10) Pub. No.: US 2012/0201893 A1
(43) Pub. Date: Aug. 9, 2012

(54) Pellets Formulation

(75) Inventors: Svenja Gutsche, Berlin (DE); Heiko Kranz, Berlin (DE); Michael Krause, Berlin (DE)

(73) Assignee: BAYER PHARMA AKTIENGESELLSCHAFT, Berlin (DE)

(21) Appl. No.: 13/384,231

(22) PCT Filed: Jul. 6, 2010
(86) PCT No.: PCT/EP2010/004113

§ 371 Date: Apr. 26, 2012

(30) Foreign Application Priority Data
Jul. 17, 2009 (EP) 09075322.9

(51) Int. Cl. A61K 9/14; A61K 9/16; A61K 31/53; A61K 9/50; A61K 31/277

(52) U.S. Cl. 424/490; 424/400; 514/523; 514/243; 264/15

(57) ABSTRACT

A process for preparing pellets by high shear granulation containing a pharmaceutical active ingredient with a pH dependent water solubility, the pellets obtained with said process and pharmaceutical oral dosage forms comprising said pellets.

### TECHNICAL FIELD

The invention relates to a process for preparing pellets containing a compound with a pH dependent solubility in water as well as to the pellets obtained with said process. The present invention further refers to oral dosage forms comprising said pellets.

Moreover, the invention also refers to a solid pharmaceutical formulation in the form of pellets or made thereof with an improved release profile comprising an active ingredient having (strong) pH-dependent water solubility, in particular said active ingredient being a weak base with low water solubility.

### BACKGROUND ART

Pharmaceutical active ingredients require to be administered in varying doses and it may be appropriate to use dosage forms or multi-unit dosage forms such as capsules or sachet. These dosage forms contain the required amounts of the active ingredient formulated in an appropriate carrier.

Pellets, commonly defined as multiple-unit dosage form, are believed to have many therapeutic advantages such as effectiveness and safety, over single-unit dosage forms. They may also be coated with a suitable coating material so to affect the release pattern of an active ingredient comprised therein.

In order to have a regular and controllable release it is required that the pellets come in regular shapes, more in particular as regularly shaped spheres.

An important factor that governs the release of an active ingredient from a pellet is the amount of the surface that is in contact with the medium to which the active ingredient is released. Irregularly shaped pellets have irregular surfaces, resulting in irregularities in the release of the active.

In this regards also the average diameter of the particles plays a key role in the release of the active and dissolution of the pellets.

The release of the active ingredient is better controllable with regularly shaped pellets having a defined average diameter. Porosity and roughness of the pellets are also parameters which influence the release of the active ingredient as well as the physical characteristics being relevant for the handling and workability of the pellets.

Spherically shaped pellets can be easily coated and a more uniform thickness of the coating can be reached when the pellets have regular round shapes. This is even more the case when the size distribution of the pellets is narrow.

Furthermore, spherically shaped pellets are easy to handle and fill into capsules, sachets or other application forms such as multiple units tablets.

Therefore, pellets used in the pharmaceutical industry are commonly free flowing, spherical particles having particle size between 0.3-2.0 mm with narrow particle size distribution and a porosity of approximately 10%.

According to the state of the art in the pharmaceutical technology field, there are several processes and equipment suitable for the production of such pellets (Isaac Ghebre-Sellassie: Pharmaceutical Pelletization Technology, Marcel Dekker, Inc., New York, Basel, 1989).

A first process developed for the production of pellets is the so-called layering method. In this process, spherical pellets of very good quality can be prepared by providing a core completed progressively by several layers of coating. The core usually comprises an inert material, e.g. sugar, starch, sodium chloride particles or a mixture thereof, and the active ingredient.

Due to the small shearing force exerted on the surface of the pellets, according to the first process above, the surface smoothness of the resulting pellets is sometimes unsatisfactory. As a consequence, the pellets binds to each other, which results in high proportion of waste. The processing time of the layering method is usually long.

A second, more important process suitable for the production of pellets is the so-called extrusion-spheronization process. In this process, the active ingredient and the required excipients, for example both in powder form, are mixed together to form a powder mixture; the appropriately homogenized powder mixture is mixed with a liquid and kneaded until a homogeneous wet mass is obtained. Said mass is extruded and the extrudate resulting from the extrusion process is spheronized (brought into a spheroid shape). Finally the raw pellets obtained from the spheronization phase are dried. The quality of the pellets is principally determined by the process variables of the extrusion and spheronization. Extrusion-spheronization processes are disclosed for example in EP1252886 and WO2007/135470.

Pellets containing microcrystalline cellulose (MCC), theophylline and a range of levels of sodium alginate (i.e., 10-50% w/w) prepared by Extrusion/Spheronization are known (Sriamornsak et al. European Journal of Pharmaceutics and Biopharmaceutics 69 (2008) 274-284). Accordingly, two types of sodium alginate were evaluated with and without the addition of either calcium acetate or calcium carbonate (0, 0.3, 3 and 10% w/w). The effects of amount and type of sodium alginate and calcium salts on pellet properties, e.g., size, shape, morphology and drug release behaviour, were investigated. The results showed that the amounts of sodium alginate and calcium salts influenced the size and shape of the obtained pellets. However, different types of sodium alginate and calcium salt responded to modifications to a different extent. A cavity was observed in the pellet structure, as seen in the scanning electron micrographs, resulting from the forces involved in the spheronization process. Most of pellet formulations released about 75-85% theophylline within 60 minutes. Incorporation of calcium salts in the pellet formulations altered the drug release, depending on the solubility of the calcium salts used.

Further processes suitable for the production of pellets (pelletization) are known in the art for example the production of pellets in high-shear mixer.

The high-shear mixer pelletization process involves distinct steps: homogenization of powders, granulation, and drying. The primer nuclei of future pellets are formed by binder spraying and dispersing during the agitation. The pellets can be finally sieved.

The formation of the pellets in the high-shear mixer is a multivariate process, therefore it is important for such a process to identify and control the process variables. In particular, the product properties are sensitive to the change of the process variables, such as impeller speed and kneading time. Hence an optimization of these parameters is critical for the process.

To avoid the development of too large particles, an appropriate agitation is required. In addition, the binder liquid flow rate can be also a critical parameter influencing quality of pellets, because the densification of agglomerates is performed under mixing and spraying. In-process control of the pellet agglomeration is crucial, hence several process indicators have to be followed and the methods to determine the end-point of the granule formation require intensive investigations.

Moreover, a number of active ingredients require specific release kinetics for example a quick and selective release of the active ingredient in a specific compartment of the gastrointestinal tract to avoid temporary over- or under-dosing of the active ingredient.

In this regard, the formulation of active ingredients belonging to class II of the biopharmaceutics classification system (Amidon et al., 1995; Dressman et al., 1998, 2001), is commonly very challenging since the oral bioavailability is determined by the dissolution rate in the gastro intestinal (GI) tract.

It is generally recognized that low solubility or dissolution rate often becomes a rate-limiting step in absorption from GI tract of pH dependent or poorly water-soluble drugs. This compromises oral bioavailability because is the concentration of drug in solution the driving force for absorption of most pharmaceutical active ingredient across biological membranes. Therefore, the enhancement of the dissolution rate of pH dependent or in general poorly water-soluble drugs after oral administration is one of the most challenging aspects of modern pharmaceutics.

According to the bioequivalence requirements established by the FDA, active ingredients with a low solubility are considered those having a solubility lower than 5 mg/ml in water (Fed. Reg. In 21CFR, Ch 1; (Apr. 1, 1987 Ed.) Part 32; 320).

Furthermore, weak base active ingredients with low solubility according to the bioequivalence requirement, usually have water solubility which is pH-dependent. These compounds usually demonstrate a relatively good solubility at lower pH-values. At higher pH (e.g. pH 6.8) the solubility of these drugs is lower than 5 mg/ml. Due to the pH-dependent solubility of the compounds pharmaceutical formulations of these drugs usually demonstrate pH dependent dissolution behaviour.

However, pharmaceutical formulations which allow a quick or extended pH independent release for low soluble active ingredients are highly desirable in the pharmaceutical field.

### DISCLOSURE OF THE INVENTION

The present invention aims to provide solid pellets comprising a pharmaceutical active ingredient, and an alginate characterised in that the mean Feret diameter is between about 300 to 800 μm, the crushing strength is between about 4-10 N and the aspect ratio is between about 1.0-1.2.

A further aim of the present invention is to provide a process for the production of solid pellets comprising a pharmaceutical active ingredient, and an alginate by high shear granulation comprising:
a mixing phase in which the respective powders of the pharmaceutical active ingredient and the required excipients are placed in a high shear mixer bowl and mixed to form a powder mixture,
a granulation phase commenced with the addition to the powder mixture of a calcium chloride solution as granulation liquid, in which the granulation mass is obtained,
a spheronization phase in which the granulation mass is spheronized by means of an impeller with the production of the pellets,
a drying phase, and
a final sieving phase.

The active ingredient comprised in the pellets according to the invention is particularly an active ingredient with a general strong pH dependent solubility, for example an active ingredient which has a good solubility at about pH 3 but with a solubility lower than 5 mg/ml at about pH 6.8 or higher. More particularly, said active ingredient is a base, for example a weak base with a pKa of about 8.5 or higher.

The pellets according to the present invention preferably comprise an alginate for example in the form of alginic acid, sodium alginate, potassium alginate, ammonium alginate, calcium alginate, magnesium alginate or a mixture thereof. Sodium alginate and calcium alginate are the most preferred.

The solid pellets according to the invention have preferably a mean Feret diameter of between about 300 to 800 μm, and a crushing strength is between about 4 to 8 N

In the process according to the present invention, the mixing phase is performed by mixing the powder comprising the active ingredient, the alginate and any other excipients in the high shear mixer bowl, for example a bowl made of glass or stainless steel. The powder is mixed for about 2 to 6 minutes with an impeller rotation of about 900 to about 1100 rpm and a chopper rotation of about 900 to about 1100 rpm.

With a bowl of about 900 ml volume and a charge of powder of about 100 g, the impeller rotation is preferably of about 1000 rpm and the chopper rotation of about 1000 rpm, for about 3 minutes.

The granulation liquid, also defined as binder, is added to the powder during the granulation phase. According to the present invention the binder is water or a water solution, preferably a calcium chloride solution or a solution of an equivalent calcium salt. Said solution has usually a calcium chloride concentration of about 3 to about 15% v/v, preferably about 5 to about 10% v/v. Instead of calcium chloride an equivalent pharmaceutical acceptable calcium salt in a correspondent required amount can be used.

The total amount of binder required for each formulation depends on the process variable like the temperature within the bowl and the impeller, as well as the concentration of the calcium salt and the final composition of the pellets.

For the conditions given above for the process according to the invention, with a charge of about 100 g, the required absolute amount of binder is between about 90 to about 130 ml with room temperature as well as heating the bowl. The temperature of the heated bowl is about 40° to 50° C. The spraying rate of the binder, which determine the volume of binding solution sprayed to the powder mixture in a minute, is usually between about 8 to 30 ml/min according the above conditions.

According to the high shear granulation process of the invention, during granulation the chopper speed remains constant, said speed being between about 2800 and 3200 rpm, preferably at 3000 rpm. The impeller speed varied between 1200 and 1400 rpm, preferably at 1300 rpm.

The spheronization phase is performed after granulation by working the pellets for about 4 to 6 minutes with an impeller speed of about 450 to about 600 rpm, preferably 500 rpm. According to a particular form of embodiment of the invention no chopper is used in the spheronization phase.

The subsequent drying phase is performed by using for example a fluidized bed coater over a period of about 5 to 20 minutes, preferably 15 minutes at a temperature of about 40 to about 55° C. (product temperature).

Finally the pellets are sieved. The pellet size fraction between a mean Feret diameter of about 300 to 800 μm, more preferably between about 400 to 600 μm are considered as acceptable pellet size range.

Solid pellets within the meaning of the present invention are pellets without a cavity.

The pellets according to the invention are produced by High Shear Granulation preferably on water basis. Compared to conventional pellets prepared for example by Extrusion/Spheronization, the pellets according to the present invention are solid and comprise a surface with a controlled roughness, as well as a low porosity and a higher crushing strength. These features confer an improved mechanical stability of the pellets and allow their homogenous and stable coating. Moreover, since they are solid a high amount of active ingredient can be loaded in said pellets.

As mentioned above, according to a particular form of embodiment of the invention, the pellets are coated. A typical coating is for example a polyvinyl acetate/polyvinyl pyrrolidone copolymer (Kollicoat SR 30 D).

The small particle size of the pellets according to the invention allows to advantageously fill a capsule with a higher amount of pellets per capsule with the effect that the dosage variability per capsule is reduced. A further advantage of the small size of the pellets is that paediatric pharmaceutical preparations can be produced.

The pellets of the present invention improve the release of the pharmaceutical active ingredient comprised therein. In particular, an independent pH release of the active ingredient comprise in the pellets according to the invention is achieved according to the invention.

Said active ingredient has for example a strong pH dependent solubility, for example an active ingredient having a good solubility at a low pH, for example at a pH of about 3, but with a solubility lower than 5 mg/ml at a high pH, for example at a pH of about 6.8 or higher.

According to a particular form of embodiment said active ingredient is a weak base with a pKa of about 8.5 or higher. This effect is particularly relevant when the pH in the dissolution medium is about 6.8 or higher and said weak base has a low solubility in water like for example in the case of vardenafil hydrochloride or verapamil hydrochloride.

An active ingredient with a strong pH dependent solubility being a weak base with a pKa of about 8.5 or higher is also considered can be also favourably comprised in the pellets according to the invention.

Pharmaceutical active ingredients which can be favourably comprised in the pellets according to the present invention are for example beta-blockers like propranolol, metoprolol and atenolol, calcium antagonists like diltiazem and verapamil, antimicrobiologcal agents like cefalexin, cefaclor, antihistamines like chlorpheniramine, cinnarizine, diphenhydramine, tranquilizers like diazepam or antipsychotics like chlorpromazine, fluphenazine, as well as any pharmaceutical acceptable salts thereof.

The improvement of the dissolution rate for the pharmaceutical active ingredient is an effect obtained by the use of the alginate. The alginate component in the pellet is better soluble at high pH values (e.g. pH=6.8) than at a low pH values (e.g. pH=3). This has the effect that the dissolution of the weak base as pharmaceutical active ingredient is retarded at a low pH (e.g. pH=3), due to the slow disintegration of the pellets, while this is improved at high pH values (e.g. pH=6.8), by the dissolution of the active ingredient and the fast disintegration of the pellets.

The use of the alginate allows an improved dissolution rate of the active ingredient, at high pH values (e.g. pH 6.8) where the dissolution is commonly limited.

As a consequence of the improved dissolution rate a better bioavailability of the active ingredient is also achieved by the pellets according to the invention.

The pellets according to the invention can further comprise other excipients like matrix builder or fillers for an example water-soluble nonionic substance selected from the group comprising for example sucrose, mannitol, lactose, dextrose and sorbitol.

Cellulose or cellulose derivatives as additional formulating agent for influencing the mechanical strength of the pellets can also be used in the pellets according to the invention. Microcrystalline cellulose (MCC) is particularly advantageous.

The pellets according to the invention consist for example of about 1% to 40% w/w of a pharmaceutical active ingredient and the remaining 60 to 99% is made of 10% to 80% of alginate and 20% to 90% of a filler and/or a matrix builder.

### BRIEF DESCRIPTION OF THE DRAWINGS

The following examples are provided for purposes of illustration, not limitation. In summary, the pellets of this invention may be prepared by the general methods disclosed above, while the following examples disclose the preparation of representative forms of embodiment of this invention.

FIG. 1: SEM (Scanning Electron Microscope) micrographs of pellets prepared with 5% calcium chloride in the granulation liquid and a spraying rate of 8 ml/min (Formulation No. 7)

FIG. 2: SEM micrographs of pellets prepared with 5% calcium chloride in the granulation liquid and a spraying rate of 10 ml/min (Formulation No. 8)

FIG. 3a-b: SEM micrographs of pellets prepared with 5% calcium chloride in the granulation liquid and a spraying rate of 20 ml/min (Formulation No. 10)

FIG. 4: pH-independent release of verapamil hydrochloride from alginate (Protanal LF 120 M) matrix pellets (Formulation No. 10)

FIG. 5a-b: SEM micrographs of pellets prepared with 10% calcium chloride in the granulation liquid and a spraying rate of 20 ml/min (Formulation No. 16)

FIG. 6a-b: SEM micrographs of pellets prepared in a vessel which was heated during granulation/spheronization process; 40° C. (Formulation No. 14)

FIG. 7a-b: SEM micrographs of pellets prepared with 20% calcium chloride in the granulation liquid and a spraying rate of 20 ml/min (Formulation No. 17)

FIG. 8a-d: SEM micrographs of pellets prepared in a vessel which was heated during granulation/spheronization process; 50° C. (Formulation No. 14)

FIG. 9: Effect of the calcium chloride concentration on the release of verapamil hydrochloride in phosphate buffer pH 6.8 (Formulation Nos. 4, 10, 16, 17)

FIG. 10a-b: SEM micrographs of pure MCC pellets prepared with water as granulation liquid and a spraying rate of 20 ml/min (formulation No. 23)

FIG. 11: pH-dependent drug release of verapamil hydrochloride from pure MCC pellets (Formulation No. 23)

FIG. 12a-b: SEM micrographs of vardenafil hydrochloride containing pellets prepared with 5% calcium chloride in the granulation liquid and a spraying rate of 20 ml/min (Formulation No. 25)

FIG. 13: Release of a highly pH-dependent soluble compound (vardenafil hydrochloride) at pH 6.8 from alginate/MCC pellets compared to conventional MCC (Microcrystalline cellulose) pellets (Formulation Nos. 24 and 25).

FIG. 14: pH-independent drug release of verapamil HCI from coated pellets (curing 1 d at 60° C.) consisting of sodium alginate/MCC.

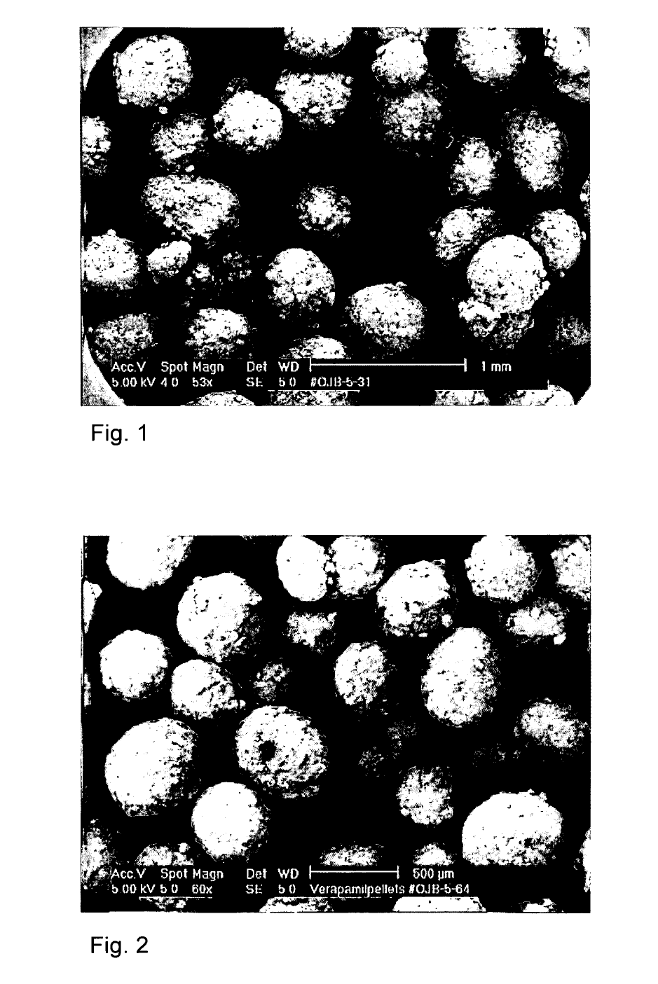

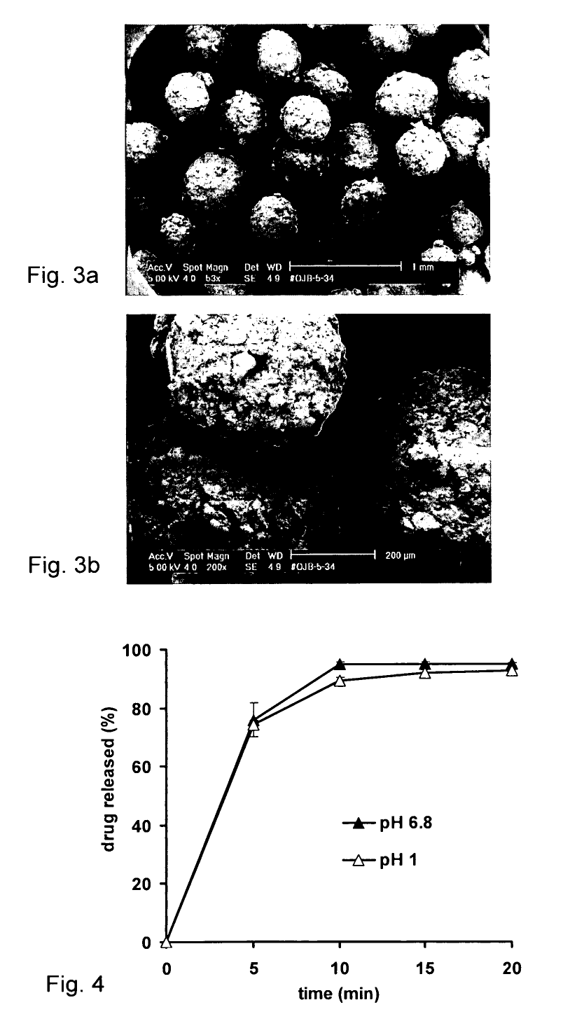

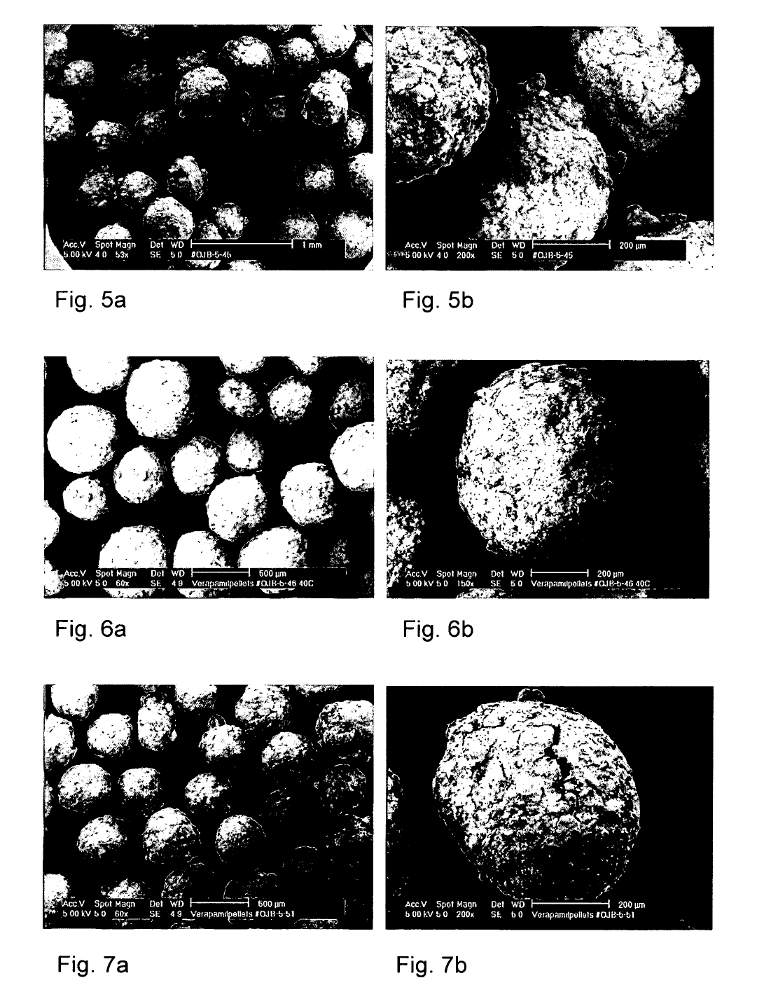

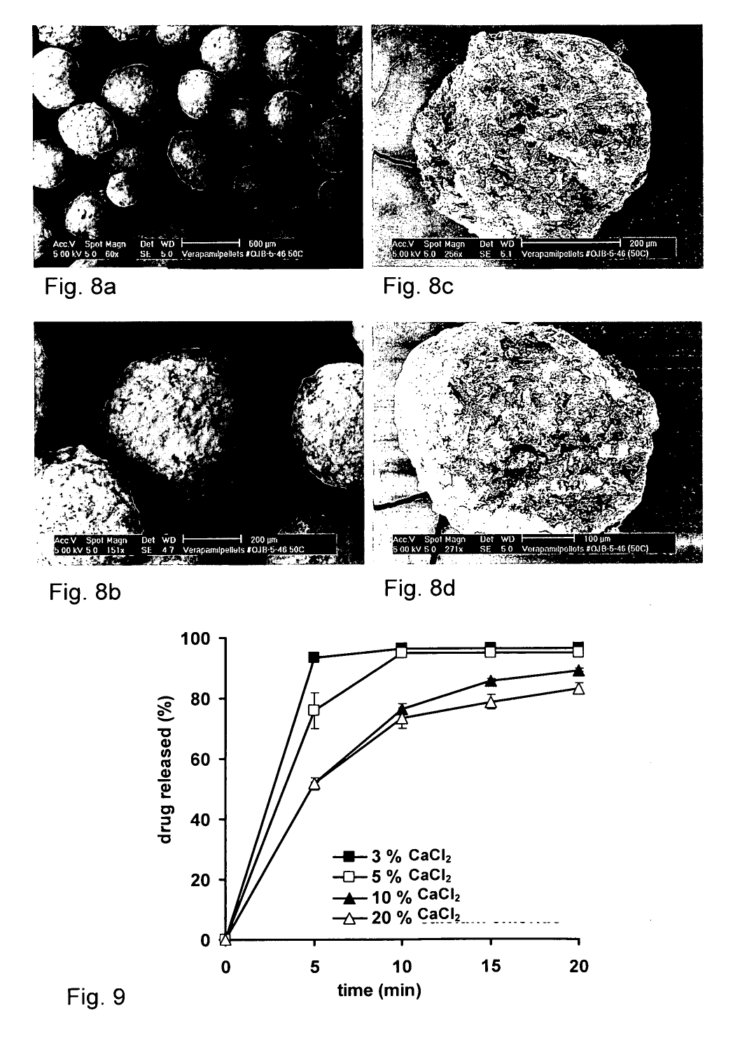

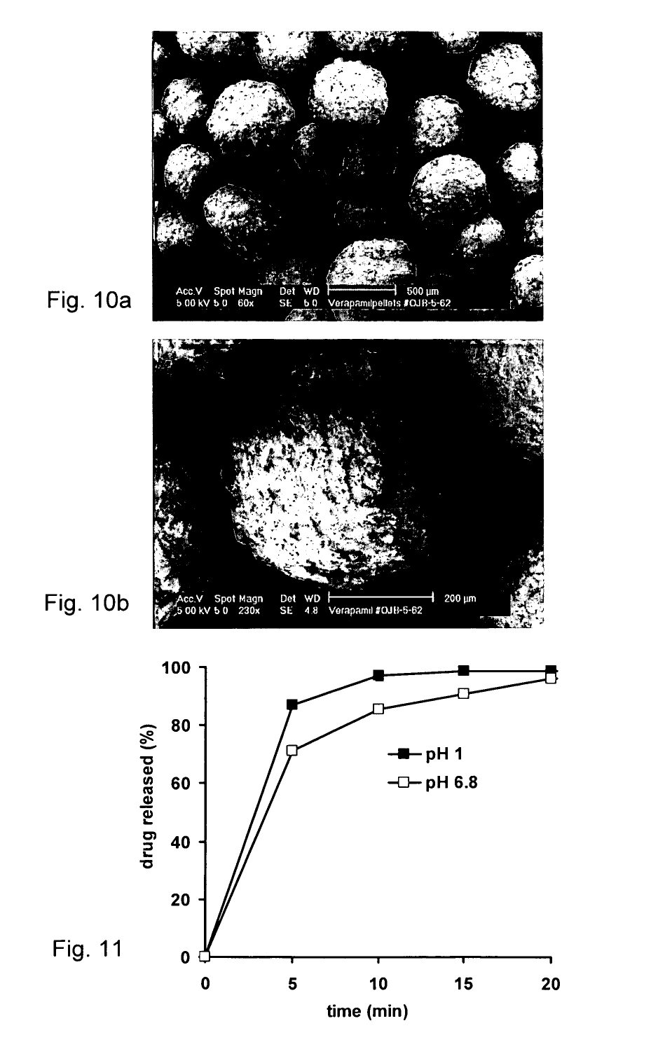

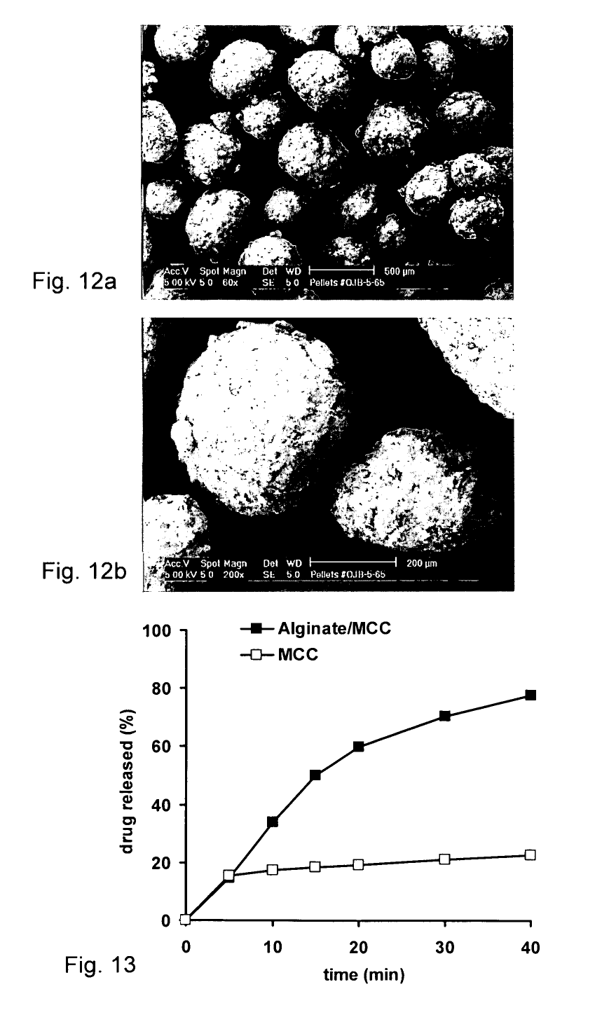

### CHARACTERIZATION METHODS

### Yield:

The yield was determined by the sieve analysis (Retsch GmbH, Haan, Germany) using the sieve apparatures of 100, 250, 355, 630, 710, 800, 900, 1000, 1400 μm. The pellet size fraction between 355-630 μm was defined as the usable yield.

pKa:

The pKa or ionisation constant is defined as the negative logarithm of the equilibrium coefficient of the neutral and charged forms of a compound. Typically in titration methods the compound is titrated towards the direction of its neutral form.

The pKa's of poorly soluble compounds can be measured in aqueous-methanol solution. If several titrations are carried out with different ratios of Methanol:Water the Yesuda-Shedlovsky equation can reveal the theoretical pKa in purely aqueous solution (Avdeef A, Corner J. E. A, Thomson, S. J. “pH-Metric Logp 3. Glass Electrode Calibration in Methanol-Water Applied to pKa Determination of Water Insoluble Subatances by Potentiometric Titration “Anal. Chem. 1993, 65, pp 42-49).

### Aspect Ratio and Mean Feret Diameter:

The Aspect Ratio y_{A} (0<y_{A}≦7) is defined by the ratio of the Minimum to the Maximum Feret Diameter y_{A}=x_{Feret min}/x_{Feret max}. It gives an indication for the elongation of the particle. Some literature also used 1/y_{A} as the definition of sphericity. Feret's Diameter is deducted from the projected area of the particles using a slide gauge. In general it is defined as the distance between two parallel tangents of the particle at an arbitrary angle. In practice the Minimum x_{F,min} and Maximum Feret Diameter x_{F,max}>, the Mean Feret Diameter x̅_{F} and the Feret Diameters obtained at 90° to direction of the Minimum and Maximum Feret Diameters X_{F,max90} are used. The minimum Feret diameter is often used as the diameter equivalent to a sieve analysis.

Both, the aspect ratio and mean Feret diameter were determined by image analysis. The sieve fractions of 355 to 630 μm (pellets prepared through the high shear granulation process) and 1000-1400 μm (pellets prepared through the extrusion/spheronisation process) were used in the image analysis. The image analysis was conducted by using an optical microscope (Olympus BX 50, Olympus Deutschland GmbH, Hamburg, Germany) combined with a video camera (HV-T20, Hitachi Kokusai Electric Europe GmbH, Erkrath, Germany). For the pellet size and shape measurements, more than 500 pellets of each sample were prepared on a glass slide, which was scanned and imaged by the video camera. The digital image processing was performed with the “Software Analysis five” (Olympus Soft Imaging Solutions GmbH, Munster, Germany). For each pellet, 36 Feret diameters were determined and used to calculate the mean Feret diameter. The ratio of the maximum Feret diameter and the Feret diameter perpendicular to the maximum Feret diameter is used as the aspect ratio.

### Porosity:

The porosity (ε) of the pellet can be calculated from the gas pycnometric density and the mercury porosimeter density according to Eqs. (1)

$$ɛ = ( 1 - \frac{{ρ}_{p}}{{ρ}_{g}} )$$

The gas pycnometric density (ρ_{g}) of the pellets was determined by a helium pycnometer (Ultrapyk 1000T, Quantachrome, Odelzhausen, Germany). For each tested pellet batch three samples were analysed.

The apparent density of the pellets (ρₚ), was evaluated by a mercury porosimeter (Pascal 140 & 440, Fisons-Carlo Erba, Valencia, USA).

### Mechanical Properties (Crushing Strength):

The crushing strength of 15 pellets was investigated by using a texture analyzer (TAXT plus, Stable Micro Systems, Godalming, Surrey UK). Each pellet was placed between the flat plate and the upper punch of the analyzer. The punch which has a diameter of 2 mm, was then lowered at a rate of 0.1 mm/sec. The point at which the pellet fractured is shown on the force time graph as the first peak. Force time plots were recorded using the texture analyzer software Exponent. The arithmetic mean of the fracture force was used as the crushing strength.

### Scanning Electron Microscopy (SEM) Pictures:

Pellets were coated for 60 s under an argon atmosphere with gold-palladium (MED 020, Bal-tec AG, Liechtenstein, Germany) and then observed with a scanning electron microscope (SEM) (DSM 982, Zeiss, Oberkochen, Germany).

### Drug Release:

In vitro release of verapamil hydrochloride was determined using the 31 USP rotating paddle method (900 ml 0.1 N HCI or USP phosphate buffer pH 6.8; 37° C.; 75 rpm; n=3; Distek Premiere 5100 Dissolution System, Distek Inc., North Brunswick, USA). At predetermined time intervals 10 ml samples were withdrawn (not replaced), filtered and assayed. The amount of the active ingredient released was measured by HPLC according to the USP 31 method.

The dissolution of vardenafil hydrochloride was performed in 900 mL phosphate buffer pH 6.8 (10%) with 0.1% sodium lauryl sulphate by using the 31 USP rotating paddle method at 75 rpm. At predetermined time intervals 10 ml samples were withdrawn (not replaced), filtered and assayed. The amount of the active ingredient released was determined by HPLC.

### Confocal Laser Scanning Microscopy (CLSM):

The CLSM method was used to determine the surface roughness value (Ra-value) of pellets said method is reported for example in F. Depypere et al., Quantification of microparticle coating quality by confocal laser scanning microscopy (CLSM), Eur. J. Pharm. Biopharm. (2009).

### High Shear Granulation:

Pellets were produced in a small scale laboratory high shear mixer (Pro-C-ept 4M8, Zelzate, Belgium) equipped with a glass bowl of 900 ml, a three blade impeller, and a chopper. The active ingredient and the required excipients for example both in powder form, were placed in the high shear mixer bowl and mixed together with an impeller rotation of 1000 rpm, and chopper rotation of 1000 rpm for 3 minutes, to form a powder mixture. Granulation commenced with the addition of a calcium chloride solution or water as a granulation liquid to from a the wet powder mass. A tube with an opening of 1 mm was used to control the rate of liquid release with a dosimat (765 Dosimat, Metrohm Ltd., Hensau, Switzerland). The total amount of granulation liquid required for each formulation was dependent on the process variables and composition of pellets. During granulation the impeller speed chosen was 1300 rpm and the chopper speed remained constant at 3000 rpm. After granulation, pellets were spheronized for 5 minutes with an impeller speed of 500 rpm (and no chopper). A fluidized bed was employed to dry out the pellets over a period of 10 minutes at 40° C. product temperature. The pellets were sieved and pellet size fraction between 355-630 μm was collected.

In case of experiment no. 14 the glass vessel was used at room temperature as well as heated during granulation. The two temperatures used in this experiment were 40° C. and 50° C.

### High Shear Granulation

### Composition of the Powder Mixture (Table 1-4):

20.0 g Verapamil hydrochloride

49.5 g Microcrystalline cellulose (Avicel PH 101)

30.5 g Natriumalginat (Protanal LF 120 M)

TABLE 1
Pellets prepared with 3% calcium chloride in the granulation liquid

| | Formulation No. | | | | | |
|---|---|---|---|---|---|---|
| | 1 | 2 | 3 | 4 | 5 | 6 |
| Spraying rate | 8 | 10 | 15 | 20 | 25 | 30 |
| (ml/min) | | | | | | |
| Absolute | 106 | 112 | 117 | 122 | 125 | 127 |
| amount binder | | | | | | |
| (ml) | | | | | | |
| Yield (%) | 61.5 | 61.5 | 67.5 | 61.5 | 58 | 70 |
| Crushing | 1.526 ± 0.68 | 2.343 ± 0.637 | 7.428 ± 1.938 | 7.887 ± 1.436 | 8.423 ± 2.148 | 8.811 ± 0.91 |
| strength (N) | | | | | | |
| Mean Feret | 0.66867 ± 0.139 | 0.66345 ± 0.142 | 0.5886 ± 0.07 | 0.55343 ± 0.08 | 0.58378 ± 0.07 | 0.54627 ± 0.07 |
| diameter (mm) | | | | | | |
| Aspect ratio | 1.29 ± 0.041 | 1.38 ± 0.102 | 1.35 ± 0.07 | 1.39 ± 0.04 | 1.4 ± 0.07 | 1.39 ± 0.05 |
| Porosity (%) | 35.12 | 27.26 | — | — | — | 10.22 |

Table 1 shows how the increase in the total amount of granulation liquid, with the calcium chloride content of the granulation liquid stable at 3%, resulted in the pellets having a higher crushing strength. This is due to the greater plasticity of the material, resulting in denser pellets with lower porosity.

The porosity values of the formulations manufactured with a low total amount of granulation liquid and a high total amount of granulation liquid were about 35% and 10%, respectively. This indicates good agreement with the hypothesis that an increase in granulation liquid leads to denser pellets with lower porosity. Consequently, pellets with lower total amounts of granulation liquid were characterized by a lower crushing strength when compared with commercially obtained pellets composed of microcrystalline cellulose, Cellets (3.8 N). However, the formulations prepared with higher total amounts of granulation liquid demonstrated good hardness with crushing strength values greater than 7.4 N.

TABLE 2
Pellets prepared with 5% calcium chloride in the granulation liquid

| | Formulation No. | | | | | |
|---|---|---|---|---|---|---|
| | 7 | 8 | 9 | 10 | 11 | 12 |
| Spraying rate | 8 | 10 | 15 | 20 | 25 | 30 |
| (ml/min) | | | | | | |
| Absolute | 107 | 112 | 114 | 115 | 116 | 117 |
| amount binder | | | | | | |
| (ml) | | | | | | |
| Yield (%) | 77.5 | 74.8 | 77.5 | 72.5 | 74.8 | 76.5 |
| Crushing | 7.089 ± 1.038 | 8.719 ± 1.453 | 7.164 ± 1.035 | 7.146 ± 1.656 | 7.262 ± 1.936 | 6.961 ± 1.274 |
| strength (N) | | | | | | |
| Mean Feret | 0.52751 ± 0.078 | 0.69844 ± 0.017 | 0.53360 ± 0.064 | 0.49059 ± 0.042 | 0.53735 ± 0.070 | 0.61309 ± 0.069 |
| diameter (mm) | | | | | | |
| Aspect ratio | 1.19 ± 0.017 | 1.23 ± 0.027 | 1.18 ± 0.016 | 1.16 ± 0.015 | 1.20 ± 0.021 | 1.18 ± 0.028 |
| Porosity (%) | | — | — | 9.57 | — | — |

TABLE 3
Pellets prepared with 10% calcium chloride in the granulation liquid

| Formulation No. | 13 | 14 | 15 | 16 |
|---|---|---|---|---|
| Spraying rate | 8 | 10 | 15 | 20 |
| (ml/min) | | | | |
| Absolute amount | 100 | 100 | 100 | 102 |
| binder (ml) | | | | |
| Yield (%) | 72.5 | 74.3 | 72.3 | 72.8 |
| Crushing | 6.312 ± 1.023 | 4.749 ± 1.516 | 5.436 ± 1.140 | 6.319 ± 1.082 |
| strength (N) | | | | |
| Mean Feret | 0.6458 ± 0.012 | 0.52547 ± 0.032 | 0.53589 ± 0.027 | 0.53079 ± 0.066 |
| diameter (mm) | | | | |
| Aspect ratio | 1.17 ± 0.013 | 1.16 ± 0.076 | 1.15 ± 0.032 | 1.17 ± 0.019 |
| Porosity (%) | — | — | — | — |

Table 2 and 3 shows that the increase in calcium chloride concentration in the granulation liquid improved the pellet shape and reduced the undesirable fraction of lumps due to the lower adhesive properties of sodium alginate caused by a dominant cross linking process. Pellets were considered acceptable, because all values for the median aspect ratio were below 1.2 with a high yield of about 72.5-77.5% (formulation nos. 7-16). The crushing strength varied between 4.7 N and 8.7 N, that indicating good hardness of all formulations, whatever spraying rate was applied. The good hardness was a result of the improved crosslinking between alginate and calcium ions.

The drug release of verapamil hydrochloride from pellets containing both MCC and sodium alginate was almost pH-independent in a range of 1 to 6.8 (FIG. 4, formulation no. 10). The alginate (Protanal® LF 120 M) was able to offset the solubility differences of the drug due to its inverse solubility. Sodium alginate dissolves more slowly at low pH levels as alginate precipitates into the form of a poorly soluble alginic acid, which creates a diffusion barrier. The faster drug release in the phosphate buffer was affected by the sodium ion exchange with the calcium ions, thus making the pellet structure to disintegrate.

TABLE 4
Pellets prepared with 20% calcium
chloride in the granulation liquid

| Formulation No. | 17 |
|---|---|
| Spraying rate (ml/min) | 20 |
| Absolute amount binder (ml) | 86 |
| Yield (%) | 60.5 |
| Crushing strength (N) | 3.659 ± 0.803 |
| Mean Feret diameter (mm) | 0.6780 ± 0.043 |
| Aspect ratio | 1.18 ± 0.023 |
| Porosity (%) | — |

Pellets prepared with calcium chloride concentrations of 20% showed cracks on the surface structure due to a non-optimized rigidity/plasticity ratio of the wet powder mass obtained from the powder mixture and the sprayed granulation liquid. The high rigidity fraction reduced the binding properties of the powder mass, and pellets consequently became instable. Therefore, the pellets prepared with 5% and 10% calcium chloride concentrations were considered optimal in relation to their properties.

As can be seen in FIG. 9 (formulation nos. 4, 10, 16, 17), the calcium concentration in the granulation liquid affected drug release rates in phosphate buffer pH 6.8. By comparing the release profiles it is evident that the rate of drug release decreased when the concentration of calcium chloride was increased. The lower release rate is connected to the increased restriction in the mobility of the polymeric chains which limits the swelling ratio of the pellets.

### Composition of the Powder Mixture (Table 5):

20.0 g Verapamil hydrochloride

40.0 g Microcrystalline cellulose (Avicel PH 101)

40.0 g Natriumalginat (Protanal LF 120 M)

TABLE 5
Pellets prepared with 5% calcium chloride in the granulation liquid

| Formulation No. | 18 | 19 | 20 | 21 | 22 |
|---|---|---|---|---|---|
| Spraying rate | 8 | 10 | 15 | 20 | 30 |
| (ml/min) | | | | | |
| Absolute amount | 107 | 114 | 118 | 121 | 122 |
| binder (ml) | | | | | |
| Yield (%) | 74.8 | 47.8 | 65.25 | 54.5 | 58 |
| Crushing | 5.774 ± 1.067 | 6.462 ± 1.111 | 7.092 ± 1.661 | 7.676 ± 2.108 | 5.614 ± 0.985 |
| strength (N) | | | | | |
| Mean Feret | 0.5961 ± 0.073 | 0.5968 ± 0.075 | 0.5315 ± 0.07 | 0.5267 ± 0.071 | 0.5347 ± 0.069 |
| diameter (mm) | | | | | |
| Aspect ratio | 1.29 ± 0.048 | 1.32 ± 0.039 | 1.2 ± 0.031 | 1.32 ± 0.055 | 1.21 ± 0.047 |
| Porosity (%) | — | — | — | — | — |

The production of pellets in a high shear granulator with an acceptable yield of 54.5-74.8% was possible, even though the alginate amount had been increased. Pellets are characterized by a good hardness with values between 5.6 and 7.7 N, but higher aspect ratio values over the range from 1.2 and 1.29 were obtained. This derives from the higher amount of alginate, which swells in contact with water. So the plasticity/rigidity properties of the granulation mass were not optimal to form spheres.

### Composition of the Powder Mixture (Table 6):

20.0 g Verapamil hydrochloride

60.0 g Microcrystalline cellulose (Avicel PH 101)

20.0 g Lactose Monohydrat (Granulac 200)

TABLE 6
Pellets prepared with water as granulation liquid

| Formulation No. | 23 |
|---|---|
| Spraying rate (ml/min) | 10 |
| Absolute amount binder (ml) | 55 |
| Yield (%) | 75.3 |
| Crushing strength (N) | 3.530 ± 0.724 |
| Mean Feret diameter (mm) | 0.5287 ± 0.048 |
| Aspect ratio | 1.18 ± 0.006 |
| Porosity (%) | — |

The characteristics of MCC based pellets are on par with those of pellets containing sodium alginate (formulation nos. 7-16).

But, verapamil hydrochloride release was slightly slower at pH 6.8 when compared to drug release rates measured at pH 1, which can be explained by the weakly basic nature of the drug (FIG. 11).

### Composition of the Powder Mixture (Table 7):

20.0 g Vardenafil hydrochloride

60.0 g Microcrystalline cellulose (Avicel PH 101)

20.0 g Lactose monohydrate

TABLE 7
Pellets prepared with water as granulation liquid

| Formulation No. | 24 |
|---|---|
| Spraying rate (ml/min) | 20 |
| Absolute amount binder (ml) | 55 |
| Yield (%) | 53.8 |
| Crushing strength (N) | 6.170 ± 2.434 |
| Mean Feret diameter (mm) | 0.6204 ± 0.061 |
| Aspect ratio | 1.22 ± 0.017 |
| Porosity (%) | — |

### Composition of the Powder Mixture (Table 8):

20.0 g Vardenafil hydrochloride

49.5 g Microcrystalline cellulose (Avicel PH 101)

30.5 g Sodium alginate (Protanal LF 120 M)

TABLE 8
Pellets prepared with 5% calcium chloride in the granulation liquid

| Formulation No. | 25 |
|---|---|
| Spraying rate (ml/min) | 20 |
| Absolute amount binder (ml) | 109.3 |
| Yield (%) | 85.8 |
| Crushing strength (N) | 4.436 ± 1.613 |
| Mean Feret diameter (mm) | 0.5291 ± 0.05 |
| Aspect ratio | 1.16 ± 0.0178 |
| Porosity (%) | — |

The characteristics of vardenafil hydrochloride pellets are on par with those of verapamil hydrochloride pellets. The use of sodium alginate in the formulation increased the yield and improved the pellet shape of vardenafil hydrochloride containing pellets.

Drug release of vardenafil hydrochloride from pure MCC based pellets was much lower than the drug release from sodium alginate/MCC based pellets at pH 6.8 (FIG. 13). Sodium alginate is more soluble in higher pH environments and therefore it increased the drug release of the poor soluble vardenafil hydrochloride in this environment.

### Extrusion/Spheronization

### Composition of the Powder Mixture (Table 9):

20.0% Verapamil hydrochloride

40.0% Microcrystalline cellulose (Avicel PH 101)

40.0% Sodium alginate (Protanal LF 120 M)

Batch size: 5000 g

TABLE 9
Pellets prepared with 3% calcium chloride in the granulation liquid

| Formulation No. | 26 |
|---|---|
| Spraying rate (ml/min) | — |
| Absolute amount binder (%) | 113.5 |
| Yield (%) | 90 |
| Crushing strength (N) | 23.63 ± 1.524 |
| Mean feret diameter (mm) | 1.0623 ± 0.05 |
| Aspect ratio | 1.08 ± 0.0024 |
| Porosity (%) | 11.53 |

The manufacture of pellets through the extrusion/spheronization method is characterized by higher processing times and complexity, when compared to the production process in the high shear granulator. Due to the adhesive properties of the extruded mass, the diameter of the perforations of the screen was increased to 1500 μm. This diameter produced pellets with a larger size when compared to pellets produced by the high shear granulation process. Hence, the mean Feret diameter of pellets prepared by extrusion/spheronization was about 0.10623 mm. The crushing strength was about 23.6 N, but the crushing strength values of the formulations prepared by extrusion/spheronization and high shear granulation can not be compared with each other because of the different pellet diameters.

Furthermore, in view of the teaching from the prior art that smaller pellets obtained by extrusion/spheronization could lead to the production of pellets in which a cavity was observed no further investigation was performed.

A relevant difference in the characteristics of the pellets obtained by high shear granulation over those obtained by extrusion/spheronization was roughness value (Ra-value) of the pellets surface. The controlled roughness of the pellets surface allows optimal flowability and workability of such multi-particulate pharmaceutical form.

The Ra-value of the pellets prepared with the Extrusion/Spheronization method was relatively low, in this case 6.7 μm for the pellets prepared according to formulation 10 of table 2, when compared to the roughness value of pellets prepared by high shear granulation according to formulation 26 of table 9 which was about 16.6 μm.

Coating of Alginate Based Pellets.

Method:

Matrix pellets containing 30.5% sodium alginate, 49.5% MCC and 20% verapamil hydrochloride were prepared by the novel high shear granulation method. To obtain optimal pellet properties for a subsequent coating process (i.e. good hardness and spherical shape), a 10% calcium chloride solution was used as granulation liquid. Pellets were then coated with an aqueous polyvinyl acetate/polyvinyl pyrrolidone dispersion (Kollicoat SR 30 D, 15% w/v solids content).

For the coating process, fractions of 40 g of pellets were coated in a fluid bed coater (Midi-Glatt, Glatt, Binzen, Germany) using bottom spray and Wurster insert until a theoretical coating level of 20% (w/w based on the core pellets) was reached.

Coating conditions: batch size: 40.0 g, inlet temperature: 30-35° C., nozzle diameter: 0.5 mm, spray pressure: 0.5 bar, spraying rate: 1 g/min and final drying at 35° C. for 5 min.

In order to avoid pellet adhesion following the coating process, silicon dioxide (Syloid 244 FP) was added to and mixed with the pellets before the curing stage. Pellets were cured for 1 d at 60° C.

Results:

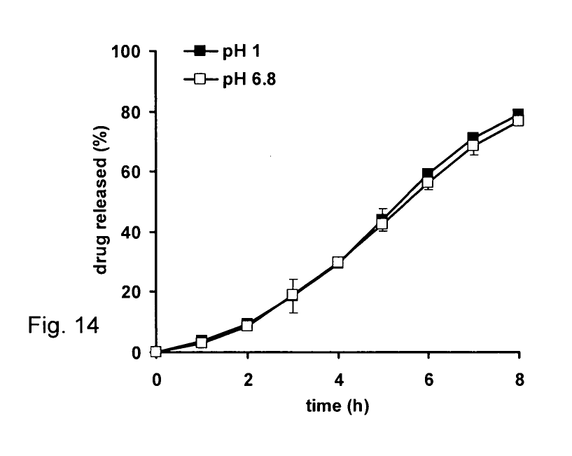

The resulting release profile of verapamil hydrochloride from coated pellets remained pH-independent (see FIG. 14).

1. A solid pellet comprising a pharmaceutical active ingredient and an alginate characterised in that the pellet has a mean Feret diameter between about 300 to 800 μm, a crushing strength between about 4 to 10 N and an aspect ratio between about 1.0 to 1.2.

2. The solid pellet according to claim 1 characterised in that the pharmaceutical active ingredient has a pH dependent solubility higher than 5 mg/ml at a pH of about 3 and lower than 5 mg/ml at a pH of about 6.8 or higher, or the pharmaceutical and/or said active ingredient has a pKa of about 8.5 or higher.

3. The solid pellet according to claim 1 characterised in that the pharmaceutical active ingredient is propranolol, metoprolol, atenolol, diltiazem, verapamil, cefalexin, cefaclor, chlorpheniramine, cinnarizine, diphenhydramine, diazepam chlorpromazine, fluphenazine, verapamil, vardenafil, or a pharmaceutical acceptable salts thereof.

4. The solid pellet according to claim 1 further comprising mannitol, lactose, dextrose, or sorbitol.

5. The solid pellet according to claim 1 further comprising cellulose or cellulose derivatives.

6. The solid pellet according to claim 1 further comprising microcrystalline cellulose.

7. The solid pellet according to claim 1 characterised in that the pellet comprises about 1% to 40% w/w of a pharmaceutical active ingredient and the remaining 60% to 99% w/w is made of 10% to 80% w/w alginate and 20% to 90% w/w filler and/or a matrix builder.

8. The solid pellet according to claim 1 characterised in that the pellet is coated.

9. A process for the production of solid pellets comprising a pharmaceutical active ingredient and an alginate by high shear granulation, the process comprising:
a mixing phase in which the respective powders of the pharmaceutical active ingredient and the alginate are placed in a high shear mixer bowl and mixed to form a powder mixture,
a granulation phase in which to the powder mixture is added a calcium chloride solution to obtain a granulation mass of solid pellets,
a spheronization phase in which the solid pellets are spheronized by means of an impeller,
a drying phase, and
a final sieving phase.

10. A process according to claim 9 in which during the mixing phase the powder mixture is mixed for about 2 to 6 minutes with an impeller rotation of about 900 to about 1100 rpm and a chopper rotation of about 900 to about 1100 rpm.

11. A process according to claim 9 in which the calcium chloride solution is in a concentration of about 3% to about 15% v/v, preferably about 5% to about 10% v/v.

12. A process according to claim 9 in which during the granulation phase the chopper speed remains constant between about 2800 and 3200 rpm, and the impeller speed is between 1200 and 1400 rpm.

13. A process according to claim 9 in which the spheronization phase is performed by working the solid pellets for about 4 to 6 minutes with an impeller speed of about 450 to about 600 rpm, preferably 500 rpm and no chopper.

14. A process according to claim 9 in which the drying phase is performed by means of a fluidized bed over a period of about 5 to 20 minutes at a temperature of about 40° to about 55° C.

15. A process according to claim 9 in which the sieving phase is performed by means of mechanical sieves.

16. A pharmaceutical oral dosage form comprising the pellets as defined according to claim 9.
